(19) United States
(12) Patent Application Publication
(10) Pub. No.: US 2004/0247697 A1
(43) Pub. Date: Dec. 9, 2004

(54) Composition with dehydration preventive ingredients

(76) Inventors: Mao-Liang Chiang, Jungli City (TW); Li-Wei Chiang, Jungli City (TW); Li-Chin Chiang, Jungli City (TW)

Correspondence Address: TROXELL LAW OFFICE PLLC, SUITE 1404, 5205 LEESBURG PIKE, FALLS CHURCH, VA, 22041, US

(21) Appl. No.: 10/453,541

(22) Filed: Jun. 4, 2003

(51) Int. Cl. A61K 35/78; A61K 33/10

(52) U.S. Cl. 424/687; 424/725; 424/739; 424/757

(57) ABSTRACT

A composition with dehydration preventive ingredients, is essentially comprised of calcium carbonate and organic substances extracted from powder of Perla Perla, Atractylis ovata Thunb, Poria cocos Wolf, Alisma plantago-aquatica L., P. Hoelen Rumph, Clcyrrhiza glabra L., Cinnamomum loureiru Nees, and saccharin to be infused in water for drink before meal to quench thirst, prevent diarrhea, alleviate food poisoning, and to avoid dehydration and make up loss of electrolytes due to vomit for alleviating discomfort.

### BACKGROUND OF THE INVENTION

(a) Field of the Invention

The present invention is related to a composition with dehydration preventive ingredients, and more particularly to one that is made of natural plants to be infused in water for drink to quench thirst, prevent diarrhea, alleviate food poisoning, and to avoid dehydration and make up loss of electrolytes due to vomit for alleviating discomfort

(b) Description of the Prior Art:

Blood circulation delivers nutrition and water containment to each part of our body. When one feels thirsty, and water is replenished in time, the blood circulation system is satisfied, and one does not feel thirsty anymore. Dehydration due to diarrhea, vomit, or long-term thirst; unbalanced water containment due to mass sweats after exercise, failure of body to absorb water containment may result in thirsty, dry mouth, dysfunction of metabolism, abnormal excretion, or serious dehydration due to poor water absorption or post-operation and excessive loss of electrolytes to endanger one's life.

### SUMMARY OF THE INVENTION

The primary purpose of the present invention is to provide a composition with dehydration preventive ingredients made of natural plants to be infused in cold water for drink to quench thirst, prevent diarrhea, alleviate food poisoning, and to avoid dehydration and make up loss of electrolytes due to vomit for alleviating discomfort. The composition may be taken before meal.

### DETAILED DESCRIPTION OF THE PREFERRED EMBODIMENTS

A composition with dehydration preventive ingredients of the present invention is essentially comprised of:

Calcium carbonate and organic substances extracted from powder Perla Perla to replenish the loss of electrolytes in one's body;

Atractylis ovata Thunb, an excitement effective for chronicle gastroenteritis, and prevention of diarrhea;

Poria cocos Wolf, a tranquilizer, essentially for treatment of vomit and diarrhea;

Alisma plantago-aquatica L., for quenching thirst, treatment of thirst, vomit, diarrhea, and gatism;

P. hoelen rumph, for diuresis, and antipyretic;

Cinnamomum loureiru Nees, conducive to the health of stomach, and as a fragrant, and an excitement;

Clycyrrhiza glabra L., as a stimulant to antidote, and add favor; and

Saccharin.

Wherein, Clycyrrhiza glabra L., Cinnamomum loureiru Nees, and saccharinare added to neutralize odors inherited to calcium carbonate and organic substances extracted from powder of Perla Perla, Atractylis ovata Thunb, Poria cocos Wolf, Alisma Plantago-aquatica L., and P. Hoelen Rumph ingredients.

The composition of the present invention is made into powder and mixed at the dosage of calcium carbonate and organic substances extracted from powder of Perla Perla (2 g), Atractylis ovata Thunb (4 g), Poria cocos Wolf (4 g), Alisma plantago-aquatica L. (5 g), P. Hoelen Rumph (4 g), Clycyrrhiza glabra L. (1 g), Cinnamomum loureiru Nees, (3 g) and saccharin (0.2 g) to be infused in cold water to drink before meal to prevent thirst and diarrhea, and to alleviate food poisoning. The composition can be taken after vomit to prevent dehydration and to replenish the lost electrolytes for alleviating discomfort.

The composition disclosed in the present invention is made of natural plants and to be infused in water for drink to prevent dehydration. Therefore this application is duly filed accordingly. However, it should be noted that the preferred embodiment is given only for reference purpose and not to limit the present invention. Any other composition with characteristics that are resembling or similar to the present invention shall be deemed as falling within the objectives and the scope of claims to be claimed by the present invention

I claim:

1. A composition with dehydration preventive ingredients is essentially comprised of:
Calcium carbonate and organic substances extracted from powder Perla Perla to replenish the loss of electrolytes in one's body;
Atractylis ovata Thunb, an excitement effective for chronicle gastroenteritis, and prevention of diarrhea;
Poria cocos Wolf, a tranquilizer, essentially for treatment of vomit and diarrhea;
Alisma plantago-aquatica L., for quenching thirst, treatment of thirst, vomit, diarrhea, and gatism;
P. hoelen rumph, for diuresis, and antipyretic;
Cinnamomum loureiru Nees, conducive to the health of stomach, and as a fragrant, and an excitement; and
Clycyrrhiza glabra L., as a stimulant to antidote, and add favor characterized by that:
the composition is to be infused in cold water for drink before mean to thirst and diarrhea, and to alleviate food poisoning. The composition can be taken after vomit to prevent dehydration and to replenish the lost electrolytes for alleviating discomfort.

2. A composition with dehydration preventive ingredients as claimed in claim 1, wherein, Saccharin is added to neutralize odors inherited to calcium carbonate and organic substances extracted from powder of Perla Perla, Atractylis ovata Thunb, Poria cocos Wolf, Alisma plantago-aquatica L., and P. Hoelen Rumph ingredients.

3. A composition with dehydration preventive ingredients as claimed in claim 2, wherein, the composition of the present invention is made into powder and mixed at the dosage of calcium carbonate and organic substances extracted from powder of Perla Perla (2 g), Atractylis ovata Thunb (4 g), Poria cocos Wolf (4 g), Alisma plantago-aquatica L. (5 g), P. Hoelen Rumph (4 g), Clycyrrhiza glabra L. (1 g), Cinnamomum loureiru Nees, (3 g) and saccharin (0.2 g).
